(19) United States
(12) Patent Application Publication
(10) Pub. No.: US 2012/0058183 A1
(43) Pub. Date: Mar. 8, 2012

(54) RETIGABINE TABLETS, PREFERABLY HAVING MODIFIED RELEASE

(75) Inventors: Sandra Brueck, Ottenhofen (DE); Katrin Rimkus, Iserlohn (DE)

(73) Assignee: ratiopharm GmbH, Ulm, Baden-Wurttemberg (DE)

(21) Appl. No.: 13/257,071

(22) PCT Filed: Mar. 17, 2010
(86) PCT No.: PCT/EP2010/001692

§ 371 Date: Nov. 22, 2011

(30) Foreign Application Priority Data
Mar. 17, 2009 (DE) 10 2009 013 612.6

(51) Int. Cl. A61K 9/28; A61K 9/20; B27N 3/02; A61K 31/27

(52) U.S. Cl. 424/465; 514/485; 264/109

(57) ABSTRACT

The invention relates to tablets, especially tablets with modified release, containing (a) retigabine and a combination of (b) water-soluble excipient and (c) non-water-soluble excipient; and a process for producing them.

The invention relates to tablets, especially tablets with modified release, containing (a) retigabine and a combination of (b) water-soluble excipient and (c) non-water-soluble excipient; and a process for producing them.

The IUPAC name of retigabine [INN] is 2-amino-4-(4-fluorobenzylamino)-1-ethoxy-carbonyl aminobenzene. The chemical structure of retigabine is shown in formula (I) below:

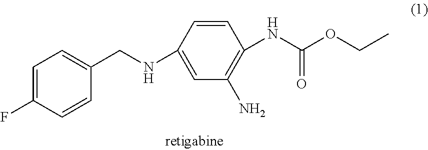

Synthesis pathways for retigabine and its use as an anti-epileptic agent have been described in EP 0 554 543. The use of retigabine for the treatment of neuropathic pain is also known from WO 01/22953 A2.

Epilepsy is one of the commonest neurological disorders and affects up to about 1% of the population. Whereas a majority of epilepsy patients can be treated with anticonvulsants currently available on the market, about 30% of patients are pharmacoresistant. There is therefore a need to develop new anticonvulsants with innovative mechanisms of action. As a potassium channel opener, retigabine, an anticonvulsant substance, satisfies these criteria. As yet, however, no pharmaceutical dosage forms are known in the art which permit an advantageous, oral administration of retigabine in high doses, especially with modified release, for the treatment of epilepsy.

WO 02/80898 A2 proposes formulating retigabine in the form of hard gelatine capsules containing 50, 100 and 200 mg active agent. Hard gelatine capsules are often felt by patients to be unpleasant to take. In particular, it is problematic to obtain a high content of active agent (e.g. 70%) in the capsule with this method. It has also become apparent that capsules produced by means of the wet granulation of retigabine are not ideal with regard to their pharmacokinetic properties and do not permit modified release.

In addition, delayed-release retigabine formulations are proposed in WO 01/66081 A2 which were produced by melt granulation, where a composition consisting solely of retigabine and sucrose fatty acid ester was used. The use of large amounts of sucrose fatty acid ester is often undesirable, however, because of the emulsifier effect. Furthermore, the proposed formulations frequently lead to an undesirably slow onset of action.

The objective of the present invention was therefore to overcome the above-mentioned disadvantages.

One object of the invention is to provide a dosage form which is pleasant for the patients and which makes it possible also to administer quantities of active agent of considerably more than 200 mg in an advantageous manner, especially also with modified release.

The aim here is especially to achieve both a rapid onset of action and also a long sustained release (and hence a constant plasma level).

The intention is to provide the active agent in a form which possesses good flowability—despite any possible micronisation—and makes good compression possible. The resulting tablets should exhibit a high level of hardness and low friability.

In particular, it is an object of the invention to provide a process for the preparation of tablets containing retigabine which exhibit advantageous lacquer coatability. During lacquer coating of the tablets of the invention, it is intended that no spalling should occur.

While developing retigabine formulations, the inventors of the present application were also confronted with the fact that crystalline retigabine can exist in different polymorphous forms. As described in WO 98/31663, these polymorphs are frequently not stable, however, but tend to change into different polymorphous forms. The frequently used retigabine hydrochloride form A, for example, can change into form B under the influence of heat. However, the polymorphous forms A, B and C have different solubility profiles.

In a patient, the different solubility profile leads to an undesirable, uneven rise in the concentration of the active agent. It is therefore an object of the present invention to provide stable retigabine intermediates that can be processed into a dosage form which enables as even a rise as possible in the concentration in the patient. The aim is largely to avoid both inter-individual and also intra-individual deviations.

In view of the lability of the active agent, it is therefore a further object to provide tablets that exhibit good storage stability.

All the objects mentioned above are supposed to be achieved in particular for a high content of active agent (drug load).

It has unexpectedly been found that the objects can be achieved by the combination of a water-soluble and a non-water-soluble excipient.

The subject matter of the invention is therefore a tablet containing

(a) retigabine,
(b) a water-soluble excipient; and
(c) a non-water-insoluble excipient.

The subject matter of the invention is also a process for producing the tablets of the invention, comprising the steps of

(I) providing (a) retigabine, (b) water-soluble excipient and (c) non-water-soluble excipient and optionally (d) disintegrant,
(II) optionally compacting them into a slug;
(III) optionally granulating the slug;
(IV) compressing the granules into tablets;
(V) optionally film-coating the tablets.

Finally, a subject matter of the invention is the use of a combination of water-soluble and non-water-soluble excipients for the production of a retigabine tablet with modified release.

In the context of this invention, the term “retigabine” (=component (a)) comprises 2-amino-4-(4-fluorobenzylamino)-1-ethoxycarbonyl aminobenzene according to the above formula (I). In addition, the term “retigabine” comprises all the pharmaceutically acceptable salts, hydrates and solvates thereof.

The salts may be acid addition salts. Examples of suitable salts are hydrochlorides (e.g. monohydrochloride, dihydrochloride), carbonates, hydrogen carbonates, acetates, lactates, butyrates, propionates, sulphates, methane sulphonates, citrates, tartrates, nitrates, sulphonates, oxalates and/or succinates. Retigabine is preferably used in the context of this invention in the form of the free base. Alternatively, retigabine is preferably used in the context of this invention in the form of the dihydrochloride.

In the context of this invention, retigabine can be used in both amorphous and crystalline form. Similarly, retigabine can also be used in the form of a solid solution It is preferable to use crystalline retigabine.

According to WO 98/31663, crystalline retigabine may be present in three different polymorphous forms (polymorphous forms A, B and C). In the context of this invention, in the case of crystalline retigabine, the polymorphous form A is preferably used.

The water-soluble excipient (=component (b)) is generally a pharmaceutical excipient specified in the European Pharmacopoeia which exhibits a water-solubility of less than 33 mg/ml, measured at 25° C. The water-soluble substance preferably exhibits a solubility of more than 50 mg/ml, even more preferably more than 100 mg/ml, especially more than 250 mg/ml, such as a water-solubility of between 250 mg/ml and 1 g/ml. Water-solubility is determined in the context of this invention using the column elution method in accordance with EU Directive DIR 67-548 EEC, Annex V, Chap. A6.

In a preferred embodiment, the water-soluble excipient (b) is a polymer, particularly preferably a hydrophilic polymer. Furthermore, the term “water-soluble excipient” (b) comprises solid, non-polymeric compounds which preferably contain polar side groups and exhibit the above-mentioned solubility. Examples of these are sugar alcohols.

The water-soluble polymer (b) used in the context of this invention is preferably a polymer which has a glass transition temperature (Tg) higher than 15° C., more preferably 40° C. to 150° C., especially 50° C. to 110° C.

The term “glass transition temperature” (Tg) is used to describe the temperature at which amorphous or partially crystalline polymers change from the solid state to the liquid state. In the process, a distinct change in physical parameters, e.g. hardness and elasticity, occurs. Below the Tg, a polymer is usually glassy and hard, whereas above the Tg, it changes into a rubber-like to viscous state. The glass transition temperature is determined in the context of this invention by means of dynamic differential scanning calorimetry (DSC). For this purpose a Mettler Toledo DSC 1 apparatus, for example, can be used. The work is performed at a heating rate of 1-20° C./min, preferably 5-15° C./min, and at a cooling rate of 5-25, preferably 10-20° C./min.

In addition, the polymer which can be used as a water-soluble polymer (b) preferably has a weight-average or number-average molecular weight of 1,000 to 100,000 g/mol, more preferably 4,000 to 70,000 g/mol, especially 5,000 to 50,000 g/mol. When the water-soluble polymer (b) is dissolved in (distilled) water in an amount of 2% by weight, the resulting solution preferably has a viscosity of 0.1 to 8 mPa×s, more preferably 0.3 to 6 mPa×s, especially 0.5 to 4 mPa×s, measured at 25° C. in accordance with Ph. Eur. 6th edition, chapter 2.2.10.

Hydrophilic polymers are preferably used, as described above, as the water-soluble component (b). This refers to polymers which possess hydrophilic groups. Examples of suitable hydrophilic groups are hydroxy, alkoxy, acrylate, methacrylate, sulphonate, carboxylate and quaternary ammonium groups.

The water-soluble excipient (b) may comprise the following polymers, for example: polysaccharides, such as hydroxypropyl methyl cellulose (HPMC), carboxymethyl cellulose (CMC, especially sodium and calcium salts), hydroxyethyl cellulose, ethyl hydro-oxyethyl cellulose, hydroxypropyl cellulose (HPC); guar flour, alginic acid and/or alginates, pectin, gum traganth; synthetic polymers such as polyvinyl pyrrolidone (povidone), polyvinyl acetate (PVAC), polyvinyl alcohol (PVA), polymers of acrylic acid and their salts, polyacrylamide, derivatives of polymethacrylates (Eudragit® E, Eudragit® R, Eudragit® S), vinyl pyrrolidone/vinyl acetate copolymers (such as Kollidon® VA64, BASF), polyalkylene glycols, such as polypropylene glycol or preferably polyethylene glycol, co-block polymers of polyethylene glycol, especially co-block polymers of polyethylene glycol and polypropylene glycol (Pluronic®, BASF), and mixtures of the poly-mers mentioned. Dextrins can also be used.

Alternatively, the component (b) may also include solid, non-polymeric compounds which preferably contain polar side groups. Examples of these are sugar alcohols or disaccharides. Examples of suitable sugar alcohols are mannitol, sorbitol, xylitol, isomalt, glucose, fructose and mixtures thereof. The term “sugar alcohols” in this context also includes monosaccharides.

In addition, fatty acid derivatives such as sodium lauryl sulphate, for example, may also be used, though with the proviso that no sucrose fatty acid esters are used.

Similarly, mixtures of the above-mentioned water-soluble excipients (b) are possible.

The non-water-soluble excipient (=component (c)) is generally a pharmaceutical excipient specified in the European Pharmacopoeia which exhibits a water-solubility of less than 33 mg/ml, measured at 25° C. The non-water-soluble substance preferably exhibits a solubility of 10 mg/ml or less, more preferably 5 mg/ml or less, especially 0.01 to 2 mg/ml (determined using the column elution method in accordance with EU Directive DIR 67-548 EEC, Annex V, Chap. A6).

The component (c) is preferably a non-water-soluble polymer or a non-water-soluble pharmaceutical excipient with polymer-like properties. Component (c) preferably leads to the modified release of the active agent release. It has been found that the release profile can be influenced especially by the choice of a component (c) with an appropriate molecular weight and degree of cross-linking, with a suitable viscosity (based on a solution of component (c) in water), suitable swelling behaviour and/or a suitable glass transition or melting temperature. Alternatively, it is also possible to use a component (c) which leads to immediate release, with the proviso that the coating used is a delayed-release coating—as described below as component (e3).

The non-water-soluble polymer (c) usually has a weight-average molecular weight of 50,000 to 2,500,000 g/mol, preferably 250,000 to 2,000,000 g/mol, more preferably 350,000 to 1,500,000 g/mol. The non-water-soluble polymer (c) can be linear or preferably cross-linked. In the latter case, the non-water-soluble polymer (c) preferably exhibits a degree of cross-linking of 0.1 to 10%, especially 0.5 to 5%. (Degree of cross-linking=number of carbon atoms linked to more than one chain/total number of carbon atoms in the polymer chain).

When the non-water-soluble polymer (c) is (at least partially) dissolved in (distilled) water in an amount of 2% by weight, the resulting solution preferably has a viscosity of more than 2 mPa×s, more preferably 4 mPa×s, particularly preferably more than 8 mPa×s, especially 10 mPa×s and, for example, up to 500 mPa×s, measured at 25° C. in accordance with Ph. Eur. 6th edition, chapter 2.2.10.

The component (c) is preferably a swellable polymer or a swellable substance with polymer-like properties. The non-water-soluble polymer (c) preferably has a swelling ratio of 1.5 to 4.5, preferably 2.0 to 4.0. The swelling ratio indicates the volume in millilitres which 1 g substance, including any slime that may be adhering to it, absorbs after swelling for 4 hours in an aqueous solution. The swelling ratio is determined in accordance with Ph. Eur. 4th edition, Chapter 2.8.4.

In addition, the component (c) is preferably a polymer or a swellable substance with a glass transition temperature or a melting temperature of less than 200° C., more preferably 20° C. to 180° C., especially 30° C. to 170° C.

The term “glass transition temperature” (Tg) is used to describe the temperature at which amorphous or partially crystalline polymers change from the solid state to the liquid state. In the process, a distinct change in physical parameters, e.g. hardness and elasticity, occurs. Below the Tg, a polymer is usually glassy and hard, whereas above the Tg, it changes into a rubber-like to viscous state. The glass transition temperature is determined in the context of this invention by means of dynamic differential scanning calorimetry (DSC). For this purpose, a Mettler Toledo DSC 1 apparatus, for example, can be used. The work is performed at a heating rate of 1-20° C./min, preferably 5-15° C./min, and at a cooling rate of 5-25, preferably 10-20° C./min. The melting temperature is determined in accordance with Ph. Eur., 6th edition, chapter 2.2.9 (Open capillary method).

Examples of suitable non-water-soluble polymers (c) are acrylate-based polymers, e.g. acrylates, methacrylate derivatives (Eudragit® NE, Eudragit® RS, Eudragit® RL); cellulose derivatives such as ethyl cellulose (EC), methyl cellulose (MC), cellulose acetyl phthalate, hydroxypropyl methyl cellulose phthalate; synthetic polymers such as polyvinyl acetate, polyvinyl chloride, nylon, polyamide, polyethylene, cross-linked polyvinyl pyrrolidone and polylactides-co-glycolides. Similarly, mixtures of the above-mentioned polymers are possible. The polymers mentioned preferably possess one or more of the functional properties mentioned above (MW, cross-linking, viscosity in solution, swelling number, melting or glass transition temperature).

Microcrystalline cellulose melts at about 250° C., decomposing in the process. The use of microcrystalline cellulose does not usually lead to the modified release of the active agent. Microcrystalline cellulose is not therefore used as component (c) in the context of this invention.

For non-water-soluble substances (with polymer-like properties), it is possible to use waxes and fats. Suitable waxes or fats are solid at 25° C. Solid paraffin or bees' wax are suitable, for example. Component (c) does not, however, comprise any sucrose fatty acid ester.

In preferred embodiments of the present invention, water-soluble excipient (b) and non-water-soluble excipient (c) are used in an amount in which the weight ratio of component (b) to component (c) is 10:1 to 1:10, more preferably 5:1 to 1:5, even more preferably 4:1 to 1:2, especially 3:1 to 1:1.

It is advantageous for components (b) and (c) to be used in particulate form and for the volume-average particle size (D50) of components (b) and (c) to be less than 500 μm, preferably 5 to 200 μm.

In one possible embodiment of the present invention, retigabine is used in micronised form.

The expression “micronised retigabine” is used in the context of this invention to designate particulate retigabine, which generally has an average particle diameter of 0.1 to 200 μm, preferably 0.5 to 100 μm, more preferably 1 to 50 μm, particularly preferably 1.5 to 25 μm and especially 2 μm to 10 μm.

The expression “average particle diameter” relates in the context of this invention to the D50 value of the volume-average particle diameter determined by means of laser diffractometry. In particular, a Malvern Instruments Mastersizer 2000 was used to determine the diameter (wet measurement with ultrasound for 60 sec., 2,000 rpm, the evaluation being performed using the Fraunhofer model), and preferably using a dispersant in which the substance to be measured does not dissolve at 20° C.). The average particle diameter, which is also referred to as the D50 value of the integral volume distribution, is defined in the context of this invention as the particle diameter at which 50% by volume of the particles have a smaller diameter than the diameter which corresponds to the D50 value. Similarly, 50% by volume of the particles then have a larger diameter than the D50 value. The terms “average particle size” and “average particle diameter” are used synonymously in the context of this application.

The tablet of the invention may contain components (a), (b) and (c) in conventional quantity ratios. In a preferred embodiment, the tablet of the invention contains

(a) 25 to 75% by weight, more preferably 35 to 70% by weight, especially more than 50 to 65% by weight retigabine,
(b) 5 to 40% by weight, more preferably 8 to 35% by weight, especially 10 to 30% by weight water-soluble excipient and
(c) 20 to 70% by weight, more preferably 22 to 55% by weight, especially 25 to 50% by weight non-water-soluble excipient, based on the total weight of components (a), (b) and (c).

The tablet of the invention may consist of components (a), (b) and (c). It is, however, preferable that the tablet of the invention should additionally contain disintegrant (=component (d)). The disintegrant (d) is normally used in an amount of 1 to 10% by weight, especially 2 to 8% by weight, based on the total weight of components (a) to (d).

“Disintegrants” is the term generally used for substances which accelerate the disintegration of a dosage form, especially a tablet, after it is placed in water. Suitable disintegrants are, for example, organic disintegrants such as carrageenan, croscarmellose and crospovidone. Alkaline disintegrants can likewise be used. The term “alkaline disintegrants” means disintegrants which, when dissolved in water, produce a pH level of more than 7.0.

Suitable alkaline disintegrants are salts of alkali and alkaline earth metals. Preferred examples here are sodium, potassium, magnesium and calcium. As anions, carbonate, hydrogen carbonate, phosphate, hydrogen phosphate and dihydrogen phosphate are preferred. Examples are sodium hydrogen carbonate, sodium hydrogen phosphate, calcium hydrogen carbonate and the like.

Croscarmellose and crospovidone are preferably used as disintegrants.

The tablet of the invention may consist of components (a), (b), (c) and (d). Alternatively, however, further pharmaceutical excipients may be added. In particular, means to improve powder flowability and lubricants are added.

One example of an additive to improve powder flowability is disperse silica, e.g. known under the trade name Aerosil®. Preferably, silica is used with a specific surface area of 50 to 400 m²/g, determined by gas adsorption in accordance with Ph. Eur., 6th edition 2.9.26.

Additives to improve powder flowability are generally used in an amount of 0.1 to 3% by weight, based on the total weight of the formulation.

In addition, lubricants may be used. Lubricants are generally used in order to reduce sliding friction. In particular, the intention is to reduce the sliding friction found during tablet pressing between the punches moving up and down in the die and the die wall, on the one hand, and between the edge of the tablet and the die wall, on the other hand. Suitable lubricants are, for example, stearic acid, adipic acid, sodium steparyl fumarate, zinc stearate and/or magnesium stearate.

Lubricants are generally used in an amount of 0.1 to 3% by weight, based on the total weight of the formulation.

It lies in the nature of pharmaceutical excipients that they sometimes perform more than one function in a pharmaceutical formulation. In the context of this invention, in order to provide an unambiguous delimitation, the fiction will therefore preferably apply that a substance which is used as a particular excipient is not simultaneously also used as a further pharmaceutical excipient.

The tablet of the invention, which contains the components (a), (b), (c) and optionally (d), is preferably a tablet with modified release. In the context of this invention, the expression “modified release” means delayed release, prolonged release, sustained release or extended release. It is preferably a kinetic system that follows sustained release.

The tablet of the invention may be film-coated. In a preferred embodiment, the components (a), (b), (c), optionally (d) and optionally the further excipients described above therefore form a tablet core, the tablet core preferably being covered with a coating (=component (e)).

In general, three different coatings (e) are possible in the context of this invention:

(e1) coatings which do not influence the release of the active agent; (e2) enteric coatings; and (e3) delayed-release coatings.

Film coatings which do not influence the release of the active agent are usually water-soluble (preferably exhibiting a solubility of more than 250 mg/ml). Enteric films exhibit a pH-dependent solubility. Delayed-release film coatings are usually non-water-soluble (preferably exhibiting a solubility of less than 10 mg/ml).

For film-coating (e), macromolecular substances are generally used, such as modified celluloses, polymethacrylates, polyvinyl pyrrolidone, polyvinyl acetate phthalate, zein and/or shellack or natural gum, such as carrageenan.

Preferred examples of film formers which do not influence the release of the active agent (e1) are methyl cellulose (MC), hydroxypropyl methyl cellulose (HPMC), hydroxypropyl cellulose (HPC), hydroxyethyl cellulose (HEC), polyvinyl pyrrolidone (PVP) and mixtures thereof. The polymers mentioned usually have a weight-average molecular weight of 10,000 to 150,000 g/mol.

HPMC is preferably used, especially HPMC with a weight-average molecular weight of 10,000 to 150,000 g/mol and/or an average degree of substitution of —OCH₃ groups of 1.2 to 2.0.

Examples of enteric coatings (e2) are cellulose acetate phthalate (CAP), hydroxypropyl methyl cellulose phthalate and polyvinyl acetate phthalate (PVAP).

Examples of delayed-release coatings (e3) are ethyl cellulose (EC, commercially available as Surelease®, for example) and polymethacrylates (commercially available as Eudragit® RL or RS and L/S, for example).

The coating (e) can be free of active agent. It is, however, also possible for the coating (e) to contain active agent (a).

In a preferred embodiment, the coating (e) contains retigabine in an amount of 1 to 45% by weight, more preferably 5 to 35% by weight, especially 10 to 30% by weight, based on the total weight of the retigabine contained in the tablet. In this case, it is preferably a coating that does not influence the release of the active agent (e1).

The thickness of the coating (e) in the case of a coating with no active agent is preferably 2 to 100 μm, especially 20 to 60 μm. The thickness of the coating (e) in the case of a coating with active agent is preferably 10 to 2 μm, especially 50 to 500 μm.

Hence, in the context of this invention, an embodiment is preferable in which 1 to 45% by weight, more preferably 5 to 35% by weight, especially 10 to 30% by weight of the amount of active agent is present as an initial dose with immediate release, and 55 to 99% by weight, more preferably 65 to 95% by weight, especially 70 to 90% by weight of the amount of active agent is present as a matrix formulation with delayed release.

The pharmaceutical formulation of the invention is preferably pressed into tablets. In the state of the art, wet granulation is proposed for this purpose (see WO 02/080898).

In principle, wet granulation is also suitable for the production of the tablets of the invention.

It has, however, become apparent that the properties of the resulting tablets can be improved if wet granulation is avoided.

The intermediates of the invention are therefore compressed into tablets by means of direct compression or are subjected to dry granulation before being compressed into tablets.

A further aspect of the present invention therefore relates to a dry-processing process comprising the steps of
(I) preparing the components (a), (b), (c) and optionally (d);
(II) optionally compacting them into a slug;
(III) optionally granulating the slug;
(IV) compressing the slug into tablets;
(V) optionally film-coating the tablets.

In step (I), the intermediate of the invention and excipients are preferably mixed. The mixing can be performed in conventional mixers. Alternatively, it is possible that the retigabine intermediate is initially only mixed with part of the excipients (e.g. 50 to 95%) before compacting (II), and that the remaining part of the excipients is added after the granulation step (III). In the case of multiple compacting, the excipients should preferably be mixed in before the first compacting step, between multiple compacting steps or after the last granulation step.

In step (II) of the process of the invention, the mixture from step (I) is compacted into a slug. It is preferable here that it should be dry compacting, i.e. the compacting is preferably performed in the absence of solvents, especially in the absence of organic solvents.

The compacting conditions are usually selected such that the intermediate of the invention is present in the form of a slug of compacted material, the density of the intermediate being 0.8 to 1.3 g/cm³, preferably 0.9 to 1.20 g/cm³, especially 1.01 to 1.15 g/cm³.

The term “density” here preferably relates to the “pure density” (i.e. not to the bulk density or tapped density). The pure density can be determined with a gas pycnometer. The gas pycnometer is preferably a helium pycnometer; in particular, the AccuPyc 1340 helium pycnometer from the manufacturer Micromeritics, Germany, is used.

The compacting is preferably carried out in a roll granulator.

The rolling force is preferably 5 to 70 kN/cm, preferably 10 to 60 kN/cm, more preferably 15 to 50 kN/cm.

The gap width of the roll granulator is, for example, 0.8 to 5 mm, preferably 1 to 4 mm, more preferably 1.5 to 3 mm, especially 1.8 to 2.8 mm.

In step (III) of the process, the slug is granulated. The granulation can be performed with methods known in the state of the art.

In a preferred embodiment, the granulation conditions are selected such that the resulting particles (granules) have a volume-average particle size ((D50) value) of 50 to 800 μm, more preferably 100 to 750 μm, even more preferably 150 to 500 μm, especially 200 to 450 μm.

In a preferred embodiment, the granulation is performed in a screen mill. In this case, the mesh width of the screen insert is usually 0.1 to 5 mm, preferably 0.5 to 3 mm, more preferably 0.75 to 2 mm, especially 0.8 to 1.8 mm.

The granules resulting from step (III) can be further processed into pharmaceutical dosage forms. For this purpose, the granules are filled into sachets or capsules, for example. The granules resulting from step (III) are preferably pressed into tablets (=step IV).

In step (IV) of the process, the granules obtained in step (III) are pressed into tablets, i.e. the step involves compression into tablets. Compression can be performed with tableting machines known in the state of the art.

In step (IV) of the process, pharmaceutical excipients may optionally be added to the granules from step (III).

The amounts of excipients added in step (IV) usually depend on the type of tablet to be produced and the amount of excipients which were already added in steps (I) or (II).

In the case of direct compression, only steps (I) and (IV) of the method described above are performed.

In the optional step (V) of the process of the invention, the tablets from step (IV) are film-coated. For this purpose, the methods of coating, especially film-coating, tablets which are standard in the state of the art can be employed. Reference is made to the above statements with regard to the coating materials used.

Apart from that, the explanations given above on preferred embodiments of the tablet of the invention can also be applied to the process of the invention.

Furthermore, the tableting conditions in both embodiments of the process of the invention are preferably selected such that the resulting tablets have a ratio of tablet height to weight of 0.005 to 0.3 mm/mg, particularly preferably 0.05 to 0.2 mm/mg.

The process of the invention is preferably performed such that the tablets of the invention contain retigabine in an amount of more than 200 mg to 1,000 mg, more preferably 250 mg to 900 mg, especially 300 mg to 600 mg. The subject matter of the invention thus relates to tablets containing 300 mg, 400 mg, 450 mg, 600 mg or 900 mg retigabine.

In addition, the resulting tablets preferably have a hardness of 50 to 300 N, particularly preferably 80 to 250 N, especially 100 to 220 N. The hardness is determined in accordance with Ph. Eur. 6.0, section 2.9.8.

Also, the resulting tablets preferably have a friability of less than 3%, particularly preferably less than 2%, especially less than 1%. The friability is determined in accordance with Ph. Eur. 6.0, section 2.9.7.

Finally, the tablets of the invention usually have a “content uniformity” of 95 to 105% of the average content, preferably 98 to 102%, especially 99 to 101%. (This means that all the tablets have a content of active agent of between 95 and 105%, preferably between 98 and 102%, especially between 99 and 101% of the average content. The content uniformity is determined in accordance with Ph. Eur. 6.0, section 2.9.6.

The above details regarding hardness, friability, content uniformity and release profile preferably relate here to the non-film-coated tablet.

In the case of a coating (e) with no active agent, the release profile of the tablets of the invention according to the USP method (paddle) exhibits a uniform release over time. The release curve exhibits sustained kinetics. The graph preferably shows a “slow” rise, i.e. a rise of less than 0.6-0.8% per minute. In this case (in contrast to rapid release), only a maximum of 50% of the active agent has been released after one hour.

In the case of a coating (e) containing active agent, the release profile of the tablets of the invention according to the USP method (paddle) exhibits kinetics indicating an initial dose of the active agent within 15 minutes, i.e. at least 15% of the active agent has been released after 15 minutes. After the 15 minutes, the remaining active agent will diffuse “slowly” out of the formulation, so that as of that time, release kinetics are found which follow the sustained type. After one hour, a maximum of 65% of the active agent has been released.

Consequently, the tablet of the invention makes it possible, thanks to the interaction between a water-soluble and a non-water-soluble excipient to provide an advantageous formulation for retigabine, especially one with modified release. Hence, one subject matter of the invention is the use of a combination of water-soluble and non-water-soluble excipients for the production of a retigabine tablet with modified release. In the use in accordance with the invention, the content of active agent in the tablet preferably amounts to more than 50% by weight. Apart from that, the explanations given above on preferred embodiments of the tablet of the invention can also be applied to the use in accordance with the invention.

The invention will now be illustrated with reference to the following examples.

### EXAMPLES

In all the Examples, retigabine is preferably used in the form of retigabine dihydrochloride, the amount specified referring to the amount of retigabine in the form of the free base. This means that the statement of 300 g retigabine corresponds to about 372 g retigabine dihydrochloride.

### Example 1

300 g retigabine were mixed with 200 g ethyl cellulose and 50 g polyvinyl pyrrolidone and blended for 15 minutes in a Turbula® W10B free-fall mixer. The mixture obtained was passed through a screen sized 500 μm and mixed with 2 g magnesium stearate. The mixture obtained was pressed on a Fette 102i rotary press. The tablets were compressed with a retigabine dose of 300 mg/tablet.

### Example 2

200 g retigabine were mixed for 15 minutes with 50 g hydroxypropyl cellulose and 50 g ethyl cellulose. (Turbula W10B). The mixture was screened and then mixed with 3 g zinc stearate, blended and pressed into 400 mg tablets.

### Example 3

300 g retigabine were mixed with 75 g PEG (Mw 8,000) and 75 g ethyl cellulose. The production process was analogous to Example 1 with 4 g magnesium stearate/Aerosil mixture (5:1)

The dose obtained was 600 mg per dosage form.

### Example 4

| | |
|---|---|
| Retigabine | 900 g |
| Pluronic ® | 200 g |
| Cellulose acetyl phthalate | 150 g |
| Aerosil ® | 10 g |
| Magnesium stearate | 6 g |

Retigabine and Pluronic® were mixed and screened.

The Aerosil and magnesium stearate were added, mixed again and pressed on an eccentric press (Korsch® EK0). The dose obtained was 900 mg per dosage form.

### Example 5a

| | |
|---|---|
| Retigabine | 400 g |
| Cellulose acetyl phthalate | 100 g |
| Sorbitol | 200 g |
| Aerosil ® | 10 g |
| Magnesium stearate | 3 g |

¾ of the retigabine was mixed for 10 minutes together with half a mixture of cellulose acetyl phthalate and sorbitol (1:2) and screened. ¼ of the retigabine was mixed for 10 minutes together with the other half of the cellulose acetyl phthalate/sorbitol mixture, and Aerosil and magnesium stearate were added and mixed for a further 3 minutes. The two mixtures produced were combined and homogenised for 10 minutes so that pressing could then be carried out. The dose obtained was 400 mg per dosage form.

### Example 5b

| | |
|---|---|
| Retigabine | 400 g |
| Cellulose acetyl phthalate | 150 g |
| Sorbitol | 150 g |
| Aerosil ® | 10 g |
| Magnesium stearate | 3 g |

¾ of the retigabine was mixed for 10 minutes together with half a mixture of cellulose acetyl phthalate and sorbitol (1:2) and screened. ¼ of the retigabine was mixed for 10 minutes together with the other half of the cellulose acetyl phthalate/sorbitol mixture, and Aerosil and magnesium stearate were added and mixed for a further 3 minutes. The two mixtures produced were combined and homogenised for 10 minutes so that pressing could then be carried out. The dose obtained was 400 mg per dosage form.

### Example 6

### Initial Dose in the Outer Coating

Example 2 was modified such that only ¾ of the active agent was incorporated in the core. ¼ of the active agent was blended for 3 minutes with 30 g of a standard hydroxypropyl methyl cellulose lacquer (Opadry® AMB), 10% in water, in Ultra Turrax® and sprayed onto the tablets in the form of a lacquer.

1. A tablet comprising
(a) retigabine,
(b) a water-soluble excipient, and
(c) a non-water-soluble excipient.

2. The tablet as claimed in claim 1, comprising
250 mg to 900 mg retigabine.

3. The tablet as claimed in claim 1, wherein the water-soluble excipient (b) exhibits a solubility in water of more than 50 mg/l at 25° C.

4. The tablet as claimed in claim 1, wherein the non-water-soluble excipient (c) is a polymer and exhibits a solubility in water of less than 10 mg/l at 25° C.

5. The tablet as claimed in claim 4, wherein the non-water-soluble polymer (c) has a weight-average molecular weight of more than 250,000 g/mol.

6. The tablet as claimed in claim 1, comprising
(a) 25 to 70% by weight retigabine,
(b) 5 to 40% by weight water-soluble excipient, and
(c) 25 to 70% by weight non-water-soluble excipient, based on the total weight of components (a) to (c).

7. The tablet as claimed in claim 1, further comprising
(d) a disintegrant.

8. The tablet as claimed in claim 7, wherein the tablet is one with modified release.

9. The tablet as claimed in claim 1, wherein components (a), (b), and (c) and optionally (d) form a tablet core and the tablet core is coated with a coating (e).

10. The tablet as claimed in claim 9, wherein the coating (e) comprises retigabine in an amount of 1 to 45% by weight, based on the total weight of the retigabine contained in the tablet.

11. The tablet as claimed in claim 10, wherein 1 to 45% by weight of the amount of active agent is present as an initial dose for immediate release and 55 to 99% of the amount of active agent is present as a matrix formulation for delayed release.

12. The tablet as claimed in claim 1, wherein retigabine is used in the form of the dihydrochloride.

13. A process for producing a tablet as claimed in claim 1, comprising the steps of
(I) preparing the components (a), (b), (c) and optionally (d);
(II) optionally compacting them into a slug;
(III) optionally granulating the slug; and
(IV) compressing the slug into tablets;

14. The use of a combination of water-soluble and non-water-soluble excipients for the production of a retigabine tablet with modified release.

15. The use as claimed in claim 14, wherein the content of retigabine in the tablet amounts to more than 50% by weight.

16. A process for producing a tablet as claimed in claim 13, further comprising the step of
(V) optionally film-coating the tablets.

17. The tablet as claimed in claim 7, comprising
(d) a disintegrant in an amount of 1 to 10% by weight, based on the total weight of components (a) to (d).

18. The tablet as claimed in claim 9, wherein components (a), (b), (c) and (d) form a tablet core and the tablet core is coated with a coating (e).

19. A process for producing a tablet as claimed in claim 1, comprising the steps of
(I) preparing a mixture of the components (a), (b), and (c); and
(IV) compressing the mixture into tablets.

20. A process for producing a tablet as claimed in claim 7, comprising the steps of
(I) preparing the components (a), (b), (c) and (d);
(II) compacting them into a slug;
(III) granulating the slug; and
(IV) compressing the slug into tablets; and
(V) film-coating the tablets.
